(19) United States
(12) Patent Application Publication
(10) Pub. No.: US 2008/0171792 A1
(43) Pub. Date: Jul. 17, 2008

(54) Use of highly concentrated formulations of 4-phenylbutyrate for treatment of certain disorders

(76) Inventors: Christopher Newton JOBDEVAIRAKKAM, Ewing, NJ (US); Raja Jeyakumar John Muthiah, Ewing, NJ (US); Harold H. Shlevin, Atlanta, GA (US)

Correspondence Address: KING & SPALDING LLP, 1180 PEACHTREE STREET, ATLANTA, GA, 30309-3521, US

(21) Appl. No.: 11/965,436

(22) Filed: Dec. 27, 2007

Related U.S. Application Data
(60) Provisional application No. 60/877,695, filed on Dec. 28, 2006.

(51) Int. Cl. A61K 31/192; A61P 35/00; A61P 35/02; A61P 41/00

(52) U.S. Cl. 514/570

(57) ABSTRACT

A highly concentrated preparation of sodium 4-phenylbutyrate in an aqueous medium as an alternative for present high dosage therapeutic treatments of certain disorders is provided, specifically for the treatment of spinal muscular atrophy (SMA), central nervous system (CNS) cancer, myelodysplastic syndrome (MS), acute leukemia, glioblastoma multiforme, amyotrophic lateral sclerosis (ALS), and colon cancer.

### CROSS REFERENCE TO RELATED APPLICATIONS

This application claims priority to U.S. Provisional Application 60/877,695, filed Dec. 28, 2006, the disclosure of which is hereby incorporated by reference in its entirety.

### FIELD OF THE INVENTION

This invention relates to the use of a highly concentrated preparation of sodium 4-phenylbutyrate in an aqueous medium as an alternative for present high dosage therapeutic treatments of certain disorders, specifically spinal muscular atrophy (SMA), central nervous system (CNS) cancer, myelodysplastic syndrome (MS), acute leukemia, glioblastoma multiforme, amyotrophic lateral sclerosis (ALS), and colon cancer.

### BACKGROUND OF THE INVENTION

Sodium 4-phenylbutyrate is currently being prescribed to treat urea cycle deficiency in children; it is sold in the USA under the trademark BUPHENYL (Ucyclyd Pharma, Inc., Glen Burnie, Md.), and in Europe under the trademark AMMONAPS (Orphan Europe). The urea cycle is the metabolic process by which the human body gets rid of nitrogen. There are six enzymes that take part in this process. A deficiency of any one of them upsets the process and causes excess nitrogen, in the form of ammonia, to accumulate in the body. The six urea cycle disorders are: carbamyl phosphate synthetase deficiency; n-acetylglutamate synthetase deficiency; ornithine transcarbamylase deficiency (the most common type); argininosuccinic acid synthetase deficiency (also called citrullinemia); argininosuccinase acid lyase deficiency; and arginase deficiency. Nitrogen accumulation is also present in patients with kidney or liver failure.

In children born with any of these rare enzyme deficiencies in the urea cycle, if the enzyme deficiency is severe, the condition leads to coma and death within a few days of birth. Such children are unable to excrete waste nitrogen as urea. Accordingly, the waste nitrogen accumulates as ammonium ions in the plasma leading to a condition known as hyperammonemia. Such genetic defects cannot be cured, but the condition can be treated by adherence to a life-long combination of a low protein diet and the administration of suitable medication. Presently, a combination of sodium phenylacetate and sodium benzoate is administered to children who have an N-acetylglutamine synthetase-1 deficiency, whereas sodium 4-phenylbutyrate (typically in a dosage of 450-600 mg/kg/day in three or more divided doses) is administered to children having an ornithine transcarbamoylase deficiency. In the latter treatment, the sodium 4-phenylbutyrate is converted to 2-phenylacetate, which combines with the amino acid glutamine present in the plasma and the resulting combination (or conjugate) is excreted as phenylacetylglutamine in the urine. Thus, administration of sodium 4-phenylbutyrate provides an alternative to the urea pathway as a means of excreting waste nitrogen from the body.

The above-mentioned commercially available forms of 4-phenylbutyrate, BUPHENYL in the US and AMMONAPS in Europe, are marketed as a granular powder for making a solution for oral administration to infants and young children, and as 500 mg tablets for adults and children weighing over 20 kg. The powder dosage is measured in one of three differently sized measuring spoons, which always leads to an imprecise dosage level. For example, a six year old child suffering from ornithine transcarbamoylase deficiency and weighing 19 kg has to take 3.8 g of powdered sodium 4-phenylbutyrate three times daily. The imprecise dosing measurement, and the need to mix the powder with a fluid for administration, leads to a lack of compliance in taking the prescribed dose at the required intervals. Consequently, it is invariably the case that children have to be admitted to hospital, sometime two or three times a year, because they feel nauseous, this being a first sign of hyperammonaemia caused by failure to maintain the dosing regimen. The symptom of nausea means the child patient cannot take the powder orally. Accordingly, in hospital the patient is treated with an intravenous infusion of sodium 4-phenylbutyrate (or sodium phenylacetate and sodium benzoate) to reduce the ammonium ion level to normal. When the nausea subsides, normal oral therapy is then resumed. Unfortunately, sometimes the delay in reaching a hospital leads to the patient being admitted in a hyperammonaemic coma; death may result or, on recovery, the child may be permanently brain-damaged.

Another important requirement for high dosage medications such as sodium 4-phenylbutyrate is the purity. High dosages such as 4 g per day or more require the purest of starting materials and good process control to bring all the impurities to less than 0.05% w/w.

WO 85/04805 discloses a process for waste nitrogen removal in human beings, wherein a compound having the formula Ph—CH₂—(CH₂)ₙ—COOH, wherein n is 2, such as 4-phenylbutyrate, is administered.

US Patent Application Publication No. 2004/0180962 discloses a delayed release methodology for using a low dosage of sodium 4-phenylbutyrate to treat urea cycle deficiency by compounding in a tablet form with hydroxypropylmethylcellulose and a release-controlling excipient (a release retarder or a liberation controller). However, such delayed release methodologies are not the best approach for treating this particular disease because a sufficient amount of the metabolite (phenylacetate) must be present in the plasma to react with glutamine and then be excreted as phenylacetylglutamine.

US Patent Application Publication No. 2004/0152784 describes a pharmaceutical composition of sodium 4-phenylbutyrate with effective aromatic flavoring agent and at least one synthetic sweetening agent. This disclosure provides a dry granulated pharmaceutical composition that can be dissolved in water before administration. One of the examples provides a maximum concentration of sodium 4-phenylbutyrate in the reconstituted solution of 250 mg/mL at 10° C. This reconstituted solution would require a relatively a large volume of solution for a suitable dosage, making it difficult to administer the drug to infants because of the large liquid volumes necessary upon dissolving the granules in water. Also, this particular pharmaceutical preparation is not stable biologically as it does not contain any preservative.

The '784 publication also demonstrates that the sweetening agent (potassium aspartame) is not stable in the aqueous reconstituted solution of the dry powder containing sodium 4-phenylbutyrate because it loses its sweetness when stored for more than a few weeks. The drug 4-phenylbutyrate is a very bitter-tasting compound, so loss of sweetness leads to a lack of compliance with the dosing regimen. Accordingly, additional precautions are needed when using the formulation is the '784 publication.

Sodium 4-phenylbutyrate is also useful for treating a variety of other medical indications, such as benign prostate hyperplasy, certain cancers, cystic fibrosis; HIV, spinocerebellar ataxia, kidney and liver failures, and thalasemia.

Another use for sodium phenylbutyrate is to induce fetal hemoglobin production in patients with sickle cell anemia; this has been described by George J. Dover (Blood, vol. 84, No. 1, Jul. 1, 1994: pp 339-343). This paper states that sodium phenylbutyrate in powdered form has a bitter taste that, despite many attempts, cannot be disguised. Two of the four subjects treated as outpatients reported an inability to maintain compliance with their dosing regimen because of the high dosage requirements (30 to 40 tablets per day).

DE 19,810,383 describes 4-phenylbutyrate as an apoptosis-inducing agent for neoplastic therapy.

WO 9937150 describes a transcription therapy for cancer using a retinoic acid and/or an inhibitor of histone deacetylase. For this treatment, 4-phenylbutyrate is classified as a histone deacetylase inhibitor.

WO 93/07866, WO 9510271, and EP 725635 all disclose compositions and methods using phenylacetic acid (a metabolite of 4-phenylbutyrate) and its derivatives for therapy and prevention of a number of pathologies, Including cancer, AIDS, anemia, and severe beta-chain hemoglobinopathies. A number of U.S. patents describe the use of phenylacetic acid as an anticancer agent (e.g., U.S. Pat. No. 6,037,376) and as an anti-viral agent (e.g., U.S. Pat. Nos. 5,877,213 and 5,710,178).

WO 9856370 and U.S. Pat. No. 6,207,195 describe therapeutic sodium 4-phenylbutyrate containing nanospheres for the treatment of cystic fibrosis by CFTR gene therapy.

US Patent Application Publication No. 2003/0195255 describes a method of administering sodium 4-phenylbutyrate orally to treat loss of mental function associated with chronic hepatic encephalopathies, recommending a high dosage of about 200-300 mg//kg initially over one to two hours, and then divided into three equal dosages daily; for adults the dose is described as 3 to 12 g/m². With regard to the synthetic of sodium 4-phenylbutyrate and related compounds, some of the methods involve using substituted malonic esters.

WO 9901420 and WO 9503271 each describes a process of preparing substituted amino malonic acid and α-amino substituted propanoic acid from its ethyl ester. Preparation of substituted butyric acid from substituted malonic esters using various reagents is reported in several published research papers. J. Med. Chem., 47 (12), 3282-3294, 2004; Bioorg & Med Chem., 11(1), 113-121, 2003; J. Med. Chem., 46 (10), 2008-2016, 2003; Enantiomer, 7(1), 1-3, 2002; J. Med. Chem., 45 (2), 263-274, 2002; J. Het. Chem., 25(6), 1689-1695, 1988.

In addition, 4-phenylbutyrate has been shown as useful for protecting against cerebral ischemic injury. (X. Qi, et al., Mol. Pharmacol., 66(4), 899-908 (2004).)

Commercial manufacturing of 4-phenylbutyric acid involves the potential carcinogen benzene as one of the raw materials. U.S. Pat. No. 6,372,938 Burzynski et al.; J. Am. Chem. Soc., 74, 1591 (1952); J. Am. Chem. Soc. 74; 4721 (1952); Bull. Acad. Sci. USSR Div. Chem. Sci. (Engl. Transl.), EN, 36, 2, 327-330 (1987); Akad. Nauk SSSR Ser. Khim.; RU, 2; 367-371 (1987).

Most of the published research articles have reported that phenyl butyrate is well tolerated even at high dosage of about 20 g a day. Phenyl butyrate is found to be an efficient inhibitor of histone deacetylase (HDAC), which regulates cell growth and death. It is used in the treatment of urea cycle disorders involving deficiencies of carbamylphosphate synthetase, ornithine transcarbamylase, or argininosuccinic acid synthetase. It is indicated in patients with neonatal-onset deficiency (complete enzymatic deficiency, presenting within the first 28 days of life) and late-onset disease (partial enzymatic deficiency, presenting after the first month of life) where there is a history of hyperammonemic encephalopathy. Phenyl butyrate is found to have multiple indications including treating various types of cancer, for example, glioblastoma multiforme, acute leukemias, colon cancer, central nervous system (CNS) cancer.

Recent research suggests that sodium phenyl butyrate may treat various types of neuromuscular diseases, such as Spinal Muscular Atrophy (SMA) in children. However, treatment of patients with SMA would require sodium phenyl butyrate dose of about 500 to 600 mg/kg/day. (Neurology, web publication 2006, doi 10.1212/01. wnl. 0000249142. 82285.d6); European Journal of Human Genetics (2004) 12, 59-65; Clin. Cancer Res. 2001; 7: 2292-2300; Neuromuscul. Disord., 2004 February; 14(2):130-5. Other conditions in which a high dose administration of sodium phenyl butyrate is used include the treatment of myelodysplastic syndrome (MS or MDS), acute leukemias, and amyotrophic lateral sclerosis (ALS; commonly known as Lou Gehrig's disease). The sodium phenyl butyrate dosage range evaluated in animals for treating ALS is from about 100mg/kg/day to about 800 mg/kg/day (Journal of Neurochemistry, 2005, 93, 1087-1098). Phenyl butyrate could also be effective for treating geriatric patients with severe Parkinson's disease, as well as in delivery of digestive enzymes for children with cystic fibrosis.

Sodium 4-phenylbutyrate is a very bitter-tasting compound and so it is very difficult for patients to comply with their dosing regimen, especially children who have to take large amounts of the medicine every day. Administering such high dose of drug in powder form or in the form of tablet as required to treat these disorders is extremely stressful to the children as it is difficult to prepare the compound in a way that is palatable to children and patients, particularly children, tend not to comply with the dosing regimens that requires multiple doses given at short intervals throughout the day. Although the treatment works, non-compliance with the present dosing regimen causes incomplete treatment leading to occasional hospitalization.

There is thus a need for a treatment of certain disorders, specifically spinal muscular atrophy (SMA), central nervous system (CNS) cancer, myelodysplastic syndrome (MS), acute leukemia, glioblastoma multiforme, amyotrophic lateral sclerosis (ALS), and colon cancer that allows fewer doses to be given in a more readily compounded formulation to increase compliance in patients, particularly in children.

### SUMMARY AND OBJECTS OF THE INVENTION

Accordingly, one object of this invention is to provide an improved pharmaceutical composition containing sodium 4-phenylbutyrate for the use by patients presently administered with a high dosage and high volume dose of this drug.

In particular, in one embodiment, a method of treating patient suffering from either one or more of the indications selected from the group consisting of spinal muscular atrophy (SMA), central nervous system (CNS) cancer, myelodysplastic syndrome (MS), acute leukemia, glioblastoma multiforme, amyotrophic lateral sclerosis (ALS), and colon cancer, comprising administering an aqueous composition of sodium phenyl butyrate at a concentration of at least about 300 mg/mL to the patient in need thereof.

A process is provided that allows preparation of high liquid dosage of sodium 4-phenylbutyrate in a concentrated aqueous composition, preferably containing at least one of a preservative and a sweetening agent, and preferably both, in addition to a flavoring agent. In certain embodiments, a fragrance can also be added. The supersaturated composition can have a concentration up to 500 mg/mL of sodium 4-phenylbutyrate or more, typically the concentration ranges from about 300 mg/mL to about 700 mg/mL. A preservative such as sodium benzoate can be present, such as at about 2.5 mg/mL. In other embodiments, the dosage can include a sweetening and/or other flavoring agent, such as about 2 mg/mL of sodium saccharine or 0.01 mg/mL of sucralose. In some embodiments a flavoring agent such as raspberry or cherry is added, for example about 2 mg/mL of raspberry flavoring. This highly concentrated liquid dosage is more concentrated and more palatable, leading to easier administration to young patients and facilitating improved compliance to the dosing regimen. This concentrated solution is effective and very easy to administer to babies because it requires only a few milliliters at any one dosing time; and it is easy to administer to children because each dosage is only a few milliliters of solution at any one time.

A process of preparing a supersaturated composition of sodium 4-phenylbutyrate in water by adding sufficient water to a known quantity of sodium 4-phenylbutyrate at an elevated temperature of about 30° to about 80° C. to produce a concentration of about 600 mg/mL, or by adding the compound to a known quantity of water. The composition can be adjusted to a different pH, such as with an acid such as hydrochloric acid.

Yet another object of this invention is to provide a process for manufacturing sodium 4-phenylbutyrate with impurities at a level less than 0.05% (weight/weight basis). The general process provided by this invention is to treat Ph—(CH₂)₂—CH(COOEt)₂ (i.e., diethyl 2-phenylethylmalonate) with acetic acid and aqueous hydrochloric acid to produce 4-phenylbutyric (or 4-phenylbutanoic) acid. In another and continuing embodiment, conversion of 4-phenylbutyric acid to its sodium salt is accomplished in an organic solvent medium with an inorganic base.

In summary this invention provides a pharmaceutical liquid composition, comprising a solution of sodium 4-phenylbutyrate in an aqueous medium at a concentration of at least about 300 mg//mL, including generally at a concentration of about 300 mg/mL to about 700 mg/mL, and more preferably at a concentration of about 400 mg/mL to about 600 mg/mL. As a dosage the composition preferably further comprises at least one or more of a flavoring agent, including sweeteners, a preservative, and compatible mixtures thereof. The composition may also include an inorganic base.

A process for making a highly concentration solution of 4-phenylbutyrate by dissolving the same in water, preferably at an elevated temperature is also provided, as is a process for making 4-phenylbutyrate from 4-phenylbutyric acid by dissolving the same in an organic medium, treating with an inorganic alkali, heating, adding a second solvent to precipitate the product, and isolating/purifying the product.

This invention also provides a process for making 4-phenylbutyric acid from a diester of the formula Ph—CH₂—CH₂—CH—(COOR)₂ wherein R is an alkyl of not more than four carbons, aryl, or aralkyl wherein the alkyl portion has not more than four carbons, treating the same with a mineral acid, precipitating the product, and thereafter isolating and/or purifying the same.

This invention also provides a method of treating a patient suffering from a urea cycle deficiencies, sickle-cell anemia, cancer, or potential cerebral ischemic injury, comprising providing an oral aqueous solution of 4-phenylbutyrate having a concentration of at least about 300 mg/mL and orally administering said solution to a patient in need thereof.

The liquid dosage form disclosed in this invention overcomes the disadvantages in administering high doses in powdered or table form to children, which is important in fostering compliance with the treatment schedule; the flavoring disclosed in the present invention would be an added advantage.

### DETAILED DESCRIPTION OF THE SPECIFIC EMBODIMENTS

This invention relates to methods of use of an oral liquid pharmaceutical multiple dosage form of sodium 4-phenylbutyrate in a supersaturated solution in an aqueous medium, preferably containing at least one preservative. The drug concentration in the formulation is achieved to a maximum of about 700 mg/mL, and at 600 mg/mL the solution does not freeze at 0° C.

The method comprises administering to a patient suffering from or at risk of suffering from either one or more of the indications selected from the group consisting of spinal muscular atrophy (SMA), central nervous system (CNS) cancer, myelodysplastic syndrome (MS), acute leukemia, glioblastoma multiforme, amyotrophic lateral sclerosis (ALS), and colon cancer, an aqueous composition of sodium phenyl butyrate at a concentration of at least about 300 mg/mL. In certain embodiments, a method of treating a patient suffering from a urea cycle deficiencies, sickle-cell anemia, cancer, or potential cerebral ischemic injury, comprising providing an oral aqueous solution of 4-phenylbutyrate having a concentration of at least about 300 mg/mL and orally administering said solution to a patient in need thereof is provided.

In certain embodiments, the patient being treated is a child. For purposes of this disclosure, a child is an individual under the age of 18. In other embodiments, the patient is not a child. In certain embodiments, the patient is between 1 and 10 years old. In specific embodiments, the patient is under 5 years old.

In certain embodiments, the composition administered to the patient comprises a preservative, a flavoring agent, a fragrance, or a mixture thereof. The composition can also further comprise a preservative and a flavoring agent. The composition can also further comprise a fragrance and a sweetener as the flavoring agent.

In one embodiment, a pharmaceutical liquid composition is provided comprising sodium 4-phenylbutyrate in an aqueous medium at a concentration of at least about 300 mg/mL. In certain embodiments, the composition further comprising a preservative. The composition can also further comprise a flavoring agent. In certain embodiments, the composition comprises both a preservative and a flavor. In some embodiments, the composition comprises at least two flavoring agents and a preservative.

The composition can include sodium 4-phenylbutyrate at a concentration range from about 300 mg/mL to about 700 mg/mL. The composition can also contain sodium 4-phenylbutyrate in the range from about 400 mg/mL to about 600 mg/mL. The composition can also contain the compound at a concentration of about 500 mg/mL. In certain embodiments, the weight fraction of water is less than the weight fraction of sodium 4-phenylbutyrate.

The preservative can be sodium benzoate. In certain embodiments, the sweetening agent is sodium saccharine. In other embodiments, the sweetening agent is sucralose. The composition can comprise a mixture of sodium saccharine and sucralose.

In certain embodiments, the flavor can be selected from raspberry and cherry.

The composition can also further comprise a base. In certain embodiments, the base is sodium carbonate. The base can also be sodium hydroxide. The composition can further comprise 4-phenylbutyric acid. The composition can also further comprise sodium carbonate.

In some embodiments, the aqueous composition does not freeze at 0° C.

Thermodynamically, the solubility of a species is dependent upon temperature and the interaction between the species and the solvent through various types of intermolecular and intramolecular interactions. The solute-solvent intermolecular interactions are the prime reason for the change in solubility at different temperatures. For a true solution, at a relatively higher temperature the solute-solvent intermolecular interaction is more pronounced than at a relatively lower temperature, and thus it is typically observed the solubility of a compound soluble in a given solvent increases as the temperate increases.

In this invention it has been found that the solubility of sodium 4-phenylbutyrate has been found to be exceptionally higher than that reported in the prior art (for example, the above-mentioned publication no. 2004/0152784 reports a maximum solubility of sodium 4-phenylbutyrate of 250 mg/mL at 10° C.). This art-reported solubility is believed to pertain to the maximum solubility of the monomeric form of sodium 4-phenylbutyrate in water.

As described in more detail below, a process has been identified of preparing a highly concentrated solution of sodium 4-phenylbutyrate, having a concentration 500 mg/mL in water by dissolving 5 g of sodium 4-phenylbutyrate in about 3.5 mL water to yield a solution volume of about 10 mL. The temperature can be room temperature (25° C.) or an elevated temperature, preferably in the range of up to about 80° C. The solution can be made at a higher temperature and then cooled to room temperature without precipitating resulting. The solution thus made is believed to be a supersaturated, non-ideal solution that does not obey the van't Hoff equation (a plot of −1n K versus 1/T giving a straight line, where K is the solubility constant and T is the absolute temperature). While not desirous of being constrained to a particular theory, these results suggest to us that that the solution so formed is a micellar kinetic phase where sodium 4-phenyl butyrate is the micelle in an aqueous bulk phase.

Therefore, due to likely micelle formation of sodium 4-phenylbutyrate (which we term the self-associated polymeric form), the high concentration of about 500 mg/mL can be achieved in solution. Even further, this high concentration solution did not freeze or precipitate out upon storage, even at 0° C. for two days, and only on further cooling to −40° C. is precipitation observed. This novel invention thus provides a dosage form better able to help the patients presently administered with a high volume dosage of sodium 4-phenylbutyrate. This invention is not intended to be limited by this discussion of micellar phases, or the presence or absence of other high concentration phases (such as sponge or L3, worm-like micelles, sheets and other laminar phases) that may be formed depending on the particular processing conditions and/or materials used. In the follow description the term “solution” is used without regard to whether a micellar phase is present.

In certain embodiments, a process for preparing an aqueous solution of 4-phenylbutyrate is provided, comprising the steps of: adding water to sodium 4-phenylbutyrate powder; and dissolving the powder in the water by agitation at temperature ranging from about 25° C. to about 80° C. to obtain a solution having a concentration of at least about 300 g/mL of 4-phenylbutyrate. In certain embodiments, the weight fraction of water in the solution is less than the weight fraction of 4-phenylbutyrate.

In certain embodiments, the solution does not freeze at 0° C. In specific embodiments, the process further comprising adjusting the pH to between about 7.0 and 10.0 by the addition of a pharmaceutically acceptable base and/or acid.

A process is also provided for making of sodium 4-phenylbutyrate, comprising the steps of:

(A) dissolving 4-phenylbutyric acid in a first organic solvent medium;

(B) treating the solution of step (A) with a inorganic alkali;

(C) heating the treated solution of step (B) to a predetermined temperature;

(D) adding a second solvent to the heated mixture effective to precipitate sodium 4-phenylbutyrate therefrom; and

(E) isolating the precipitate product by filtration and drying under vacuum at a predetermined temperature.

The process can further comprise concentrating the solution obtained after step (C) by distilling out the organic solvent medium. In certain embodiments, the inorganic alkali is sodium carbonate. In other embodiments, the inorganic alkali is sodium hydroxide. The process can be one wherein the first organic solvent comprises two or more organic solvents. The first organic solvent can be selected from the group consisting of methanol, ethanol, isopropanol, ethyl acetate, tetrahydrofuran, and compatible mixtures thereof. The process can also be one wherein the second solvent is two or more organic solvents. The second solvent can be selected from the group consisting of Isopropyl ether, diethylether, ethyl acetate, ethyl methyl ketone, 1,4-dioxan, acetone, and compatible mixtures thereof. In certain embodiments, the predetermined temperature in each of step (C) and (E) is independently selected to be in the range of about 30° C. to about 95° C.

A process is also provided for making 4-phenylbutyric acid, comprising:
(i) treating an organic ester of the formula Ph—CH₂—CH₂—CH—(COOR)₂, wherein each R is independently an alkyl containing up to four carbon atoms, an aryl group, or an aralkyl group wherein the alkyl portion has up to four carbon atoms, with a mineral acid in a water miscible organic solvent at a predetermined temperature; and
(ii) precipitating 4-phenylbutyric acid using a non-polar solvent.

The process can further comprise concentrating the solution by evaporation between steps (i) and (ii). The process can also further comprise step of purifying the crude 4-phenylbutyric acid obtained in step (ii) by vacuum distillation. The process can also further comprise purifying the crude 4-phenylbutyric acid obtained in step (ii) by recrystallization using one or more solvents.

In certain embodiments, each R is independently methyl, ethyl or propyl. The water miscible organic solvent can contain one or more carboxylic acids having less than 4 carbons in the main chain. The carboxylic acids can be selected from propanoic acid, substituted propanoic acid, acetic acid, substituted acetic acid, and formic acid. In certain embodiments, the mineral acid is hydrochloric acid. The mineral acid can also be sulfuric acid.

In another embodiment this invention provides a process for preparing 4-phenylbutyric acid by the scheme shown below, where an organic ester is treated with an acid in a solvent, optionally concentrating the product, purifying the product, and optionally further purifying the product.

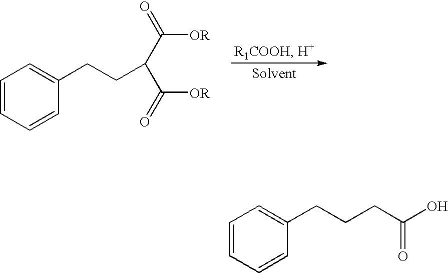

R₁=Methyl, Ethyl, Propyl, Chloromethyl, Bromomethyl,
R=Methyl, Ethyl

In this process, an organic ester of the formula Ph—CH₂—CH₂—CH—(COOR)₂ is treated with a mineral acid in a water miscible organic solvent medium at a desired temperature. Each R is independently an alkyl group containing up to four carbon atoms, or an aryl or aralkyl group wherein the alkyl portion has up to four carbon atoms. The resulting product may be concentrated, such as by evaporation (vacuum and/or temperature induced). Thereafter, the product 4-phenylbutyric acid is precipitated from solution with the aid of a non-polar solvent. This crude 4-phenylbutyric acid product may also be is purified by vacuum distillation. Finally, if desired, the crude 4-phenylbutyric acid is purified by recrystallization using a combination of non-polar solvents. In this process, the mineral acid is preferably hydrochloric acid or sulfuric acid, and the solvent contains a carboxylic acid of less than four carbon atoms in the main chain.

In another embodiment we provide a process of preparing sodium 4-phenylbutyrate including the steps of dissolving 4-phenylbutyric acid in an organic medium, treating the solution with in inorganic alkali such as sodium hydroxide or sodium carbonate, heating the resulting mixture, optionally concentrating the heated mixture by distilling out the solvent, adding a suitable solvent to the mixture to precipitate sodium 4-phenylbutyrate from the mixture, and isolating the product by filtration and drying under vacuum at a selected temperature. The organic medium is selected from one or more organic solvents preferably chosen from the group consisting of alkyl alcohols (such methanol, ethanol, and isopropanol), alkyl esters (such ethyl acetate), and tetrahydrofuran, and compatible mixtures thereof. The preferred temperature at which the solution is first heated is in the range of about 30° C. to about 95° C. In the precipitation step, the organic solvent is preferably chosen from the group consisting of dialkyl ethers (such as isopropyl ether and diethyl ether), dialkyl acetates (such as ethyl acetate), dialkyl ketones (such as acetone or ethyl methyl ketone), and other solvents, such as 1,4-dioxan, and compatible mixtures thereof.

The sodium phenyl butyrate oral solution disclosed herein can contain one or more flavorings, for example, raspberry flavor, cherry flavor, or mint flavor, in combination with a suitable sweetener to mask the bitterness of sodium phenyl butyrate.

The sodium phenyl butyrate oral solution disclosed in this invention is suitable for administering to patients, especially children, suffering from various types of Spinal Muscular Atrophy (SMA), and to treat patients with myelodysplastic syndrome (MS or MDS), and Amyotrophic lateral sclerosis (ALS).

### EXAMPLES

Practice of this invention is illustrated by the non-limiting examples provided herein.

### EXAMPLE 1

### Preparation of a Liquid Oral Pharmaceutical Composition of Sodium 4 Phenylbutyrate with a Strength of 500 mg/mL

About 12.5 g of sodium 4-phenylbutyrate was transferred to a 25 mL volumetric flask to which was added about 10 mL of water, and the mixture was agitated to dissolve the butyrate and form a solution. To the solution was added about 0.05 g of sodium saccharin, 0.05 g of sodium benzoate, and the solution was mixed well. This solution was compounded with water to yield 25 mL of a liquid oral dosage form.

### EXAMPLE 2

About 12.5 g of sodium 4-phenylbutyrate was transferred to a 25 mL volumetric flask. About 10 mL of water was added to the flask and the mixture was agitated to dissolve the butyrate. To the solution was added about 0.05 g of raspberry flavor (e.g., raspberry XBF-700194, available from IFF International Flavors & Fragrances, New York, N.Y.), 0.05 g of sodium benzoate, and then mixed well. This mixture was compounded to 25 mL with water. Any flavoring that is dispersible in water is generally suitable for this invention.

### EXAMPLE 3

About 12.5 g of sodium 4-phenylbutyrate was transferred to a 25 mL volumetric flask to which was added about 10 mL of water and agitated to dissolve. To the mixture was added about 0.05 g of sodium benzoate and mixed well. This mixture was compounded to 25 mL with water.

### EXAMPLE 4

About 12.5 g of sodium 4-phenylbutyrate was transferred to a 25 mL volumetric flask. Added about 10 mL of water and agitated to dissolve. To the mixture added about 0.05 g of raspberry flavoring, 0.05 g of sodium benzoate, 0.05 g of sodium saccharin and mixed well. This mixture was compounded to 25 mL with water.

### EXAMPLE 5

About 12.5 g of sodium 4-phenylbutyrate was transferred to a 25 mL volumetric flask, to which was added about 10 mL of water and then agitated to dissolve. To the mixture was added about 0.15 g of raspberry flavor, 0.05 g of sodium benzoate, 0.25 g of sodium saccharin and mixed well. This mixture was compounded to 25 mL with water.

### EXAMPLE 6

About 12.5 g of sodium 4-phenylbutyrate was transferred to a 25 mL volumetric flask. To that was added about 10 mL of water and the mixture agitated to dissolve. To the solution was then added about 100 mg of sodium carbonate, 0.15 g of raspberry flavor, 0.05 g of sodium benzoate, 0.25 g of sodium saccharin and mixed well. This mixture was compounded to 25 mL with water.

### EXAMPLE 7

About 12.5 g of sodium 4-phenylbutyrate was transferred to a 25 mL volumetric flask, about 10 mL of water was added, and the mixture agitated to dissolve. Then were added about: 100 mg of sodium carbonate, 0.15 g of raspberry flavor, 0.05 g of sodium benzoate, and 0.25 g of sucralose; and the combination mixed well. This mixture was compounded to 25 mL with water.

### EXAMPLE 8

About 16 g of sodium 4-phenylbutyrate was transferred to a 25 mL volumetric flask. About 9 mL of water was added and the mixture agitated with heating to a temperature of about 70° C. to dissolve. The solution was then left to cool to room temperature and about 0.05 g of raspberry flavor, 0.05 g of sodium benzoate, and 0.05 g of sodium saccharin were added with good mixing. This mixture was compounded to 25 mL with water.

### EXAMPLE 9

### Preparation of a Liquid Oral Pharmaceutical Composition of Sodium 4 Phenylbutyrate with a Strength of 640 mg/mL

About 16 g of sodium 4-phenylbutyrate was transferred to a 25 mL volumetric flask and about 9 mL of water was added and the mixture, which was then agitated with heating at a temperature of about 70° C. to dissolve the butyrate. The solution was then cooled to 25° C. and 0.05 g of sodium benzoate and 0.05 g of sodium saccharin were added with good mixing. This solution was compounded to 25 mL with water.

### EXAMPLE 10

About 16 g of sodium 4-phenylbutyrate was transferred to a 25 mL volumetric flask to which was then added about 9 mL of water. The mixture was agitated to dissolve the butyrate at an elevated temperature of about 70° C. The solution was cooled to 25° C. and 0.05 g of sodium benzoate was added and the solution mixed well. This solution was compounded to 25 mL with water.

### EXAMPLE 11

About 160 g of sodium 4-phenylbutyrate was transferred to a 250 mL volumetric flask. About 90 mL of water was added and the mixture agitated with heating at a temperature 70° C. to dissolve. The solution was then cooled to 25° C. and 0.5 g of sodium benzoate and 0.5 g of sodium saccharin were added and mixed well. This solution was compounded to 250 mL with water.

### EXAMPLE 12

About 160 g of sodium 4-phenylbutyrate was transferred to a 250 mL volumetric flask. To the flask was added about 90 mL of water and the mixture agitated with heating at a temperature 70° C. to dissolve. The mixture was cooled to 250 C and 0.5 g of sodium benzoate was added and mixed well. This mixture was compounded to 250 mL with water.

### EXAMPLE 13

About 160 g of sodium 4-phenylbutyrate was transferred to a 250 mL volumetric flask to which was then added about 90 mL of water and agitated with heating at a temperature 70° C. to dissolve. The mixture was cooled to 25° C. and 0.5 g of sodium benzoate was added and mixed well. This solution was compounded to 250 mL with water. This solution was then kept at 0° C. for about 48 hours and was no precipitation or freezing of the solution was found to have occurred. Further cooling of this solution to about −4° C. caused precipitation.

### EXAMPLE 14

### Preparation of a Liquid Oral Pharmaceutical Composition of Sodium 4 Phenylbutyrate with a Strength of 500 mg/mL Starting with 4-phenylbutyric Acid

About 10.9 g of 4-phenylbutyric acid was transferred to a 25 mL volumetric flask. About 10 mL of water was added and then about 2.9 g of sodium hydroxide was added. This mixture was agitated with heating at a temperature 70° C. for about 20 min. until a clear solution resulted. The solution was cooled to 25° C. and 0.05 g of sodium benzoate and 0.05 g of sodium saccharin were added and mixed well. This solution was compounded to 25 mL with water.

### EXAMPLE 15

About 10.9 g of 4-phenylbutyric acid was transferred to a 25 mL volumetric flask to which was added about 10 mL of water, and about 3.9 g of sodium carbonate was added. This mixture was agitated with heating at a temperature of about 90° C. for about 30 min. until a clear solution was obtained. The solution was cooled to 25° C. and then 0.05 g of sodium benzoate and 0.05 g of sodium saccharin were added and mixed well. This mixture solution compounded to 25 mL with water to provide the liquid oral composition.

### EXAMPLE 16

### Preparation of 4-phenylbutyric Acid

To a mixture of 2000 mL of acetic acid and 1500 mL of 6N hydrochloric acid was added 500 g of Diester {PhCH₂CH₂CH(COOEt)₂). The temperature of the mixture was raised to the range of about 95° to 110° C. and refluxed for about 20 hrs. The progress of the reaction was monitored by chromatography, and at completion the acetic acid and water were removed by distillation at atmospheric pressure. The residue was dissolved in water using 10% sodium hydroxide. The aqueous solution was then washed with methylene chloride and the pH was adjusted with concentrated hydrochloric acid to a pH of about 1. The product was extracted with 1700 ml of hexane and the eluate was cooled to −10° C. The resulting precipitated crude 4-phenylbutyric acid was isolated by filtration and dried under vacuum at about 30° C. Yield 280 g (90%). The crude 4-phenyl butyric acid so isolated was dissolved in 1500 mL hexane at a temperature of about 30° to 50° C. and then cooled to about −10° C. and then stirred for about one hour to precipitate. The pure 4-phenyl butyric acid was then isolated by filtration and dried under vacuum without heating. (Purity>99%.)

### EXAMPLE 17

To a mixture of 2000 mL of acetic acid and 1500 mL of 6N hydrochloric acid added 500 g of Diester {PhCH₂CH₂CH(COOEt)₂ }. The temperature of the mixture was raised to between about 95° to about 110° C. and refluxed for about 20 hrs. The progress of the reaction mixture was monitored by chromatography and at completion the acetic acid and water were removed by distillation at atmospheric pressure. The residue was dissolved in water using 10% sodium hydroxide. The aqueous solution was washed with methylene chloride and the pH was adjusted with concentrated hydrochloric acid to about one. The product was extracted with 1700 ml of hexane and the solution was cooled to −10° C. The precipitated crude 4-phenylbutyric acid was isolated by filtration and dried under vacuum at about 30° C. Yield 280 g (90%). The crude 4-phenyl butyric acid was then fractionally distilled under vacuum at about 170° C. (Purity>99%.)

### EXAMPLE 18

### Preparation of Sodium 4-phenylbutyrate

About 200 g of 4-phenylbutyric acid was dissolved in 1200 mL of methanol, then 65 g sodium carbonate was added and the mixture heated to about 60° C. for about 45 min. The solution is concentrated to about 1/10^{th} of its original volume and 7000 mL of acetone was added with stirring for about 40 min at about 0° C. The precipitated sodium4-phenylbutyrate was filtered and washed with acetone, and dried under vacuum at 30° C.

### EXAMPLE 19

### Using Sucralose as Sweetener

About 12.5 g of sodium phenyl butyrate was transferred to a 25 mL volumetric flask. And about 10 mL of warm water at about 60° C. was added; the mixture was agitated to dissolve. To the mixture were added about 0.05 g of raspberry flavor, 0.05 g of sodium benzoate, 0.05 to 0.2 g of sucralose (trichlorosucrose) and mixed well. This mixture was compounded to 25 mL with water.

### EXAMPLE 20

### Cherry Flavored

About 12.5 g of sodium phenyl butyrate was transferred to a 25 mL volumetric flask, to which was added about 10 mL of warm water at about 60° C. and agitated to dissolve. Then to the mixture were added about 0.05 g of cherry flavoring, 0.05 g of sodium benzoate, 0.05 to 0.2 g of sucralose and mixed well. This mixture was compounded to 25 mL with water.

### EXAMPLE 21

### Using Sodium Hydroxide to Adjust pH

About 12.5 g of sodium phenyl butyrate was transferred to a 25 mL volumetric flask, to which was then added about 10 mL of warm water at about 60° C. and agitated to dissolve. Then to the mixture were added about 0.05 g of cherry flavor, 0.05 g of sodium benzoate, 0.25 mg to 25 mg of sodium hydroxide to produce a pH of about 7.0 to 10.5, 0.05 to 0.2 g of sucralose, and the components were mixed well. This mixture was compounded to 25 mL with water.

### EXAMPLE 22

### Using Sodium Hydroxide/Hydrochloric Acid to Adjust pH

About 12.5 g of sodium phenyl butyrate is transferred to a 25 mL volumetric flask, and thereafter about 10 mL of warm water at about 60° C. is added; the mixture is then agitated to dissolve. Approximately 0.05 g of cherry flavor, 0.05 g of sodium benzoate, and 0.05 to 0.2 g of sucralose are added and the solution mixed well again. Adjust of the pH to between about 7.0 and about 10.5 is achieved using sodium hydroxide and/or hydrochloric acid as needed. This mixture is compounded to 25 mL with water.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A method of treating patient suffering from or at risk of one or more of an indication selected from the group consisting of spinal muscular atrophy (SMA), central nervous system (CNS) cancer, myelodysplastic syndrome (MS), acute leukemia, glioblastoma multiforme, amyotrophic lateral sclerosis (ALS), and colon cancer, comprising administering to a patient in need thereof an aqueous composition of sodium phenyl butyrate at a concentration of at least about 300 mg/mL.

2. The method of claim 1 wherein the patient is suffering from SMA.

3. The method of claim 1 wherein the concentration of the composition is between about 300 mg/ml to about 700 mg/mL.

4. The method of claim 1 wherein the concentration of the composition is between about 400 mg/ml to about 600 mg/mL.

5. The method of claim 1 wherein the composition is a micellar composition.

6. The method of claim 1 wherein the composition further comprises a flavoring agent

7. The method of claim 5 wherein the flavoring agent is cherry flavor.

8. The method of claim 1 wherein the patient is a child.

9. The method of claim 1 wherein the administration is orally.
